(19) United States
(12) Patent Application Publication
(10) Pub. No.: US 2004/0132666 A1
(43) Pub. Date: Jul. 8, 2004

(54) Administration of free radical scavengers to prevent or treat ischemia-reperfusion injuries

(75) Inventors: Edward A. Neuwelt, Portland, OR (US); Leslie Muldoon, Tigard, OR (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Oregon Health & Science University, Portland, OR

(21) Appl. No.: 10/669,158

(22) Filed: Sep. 22, 2003

Related U.S. Application Data
(60) Provisional application No. 60/412,494, filed on Sep. 20, 2002.
(60) Provisional application No. 60/478,383, filed on Jun. 13, 2003.

(51) Int. Cl. A61K 38/05; A61K 31/198; A61K 31/17

(52) U.S. Cl. 514/18; 514/562; 514/580

(57) ABSTRACT

The present invention provides methods for preventing or reducing ischemia-reperfusion injuries. These methods comprise administering an effective amount of a free radical scavenger (e.g., N-acetylcysteine) to a subject in need thereof.

### CROSS-REFERENCES TO RELATED APPLICATIONS

This application claims the benefit of U.S. Provisional Patent Application No. 60/412,494, filed Sep. 20, 2002, and U.S. Provisional Patent Application No. 60/478,383, filed Jun. 13, 2003, which provisional applications are incorporated herein by reference in their entireties.

### STATEMENT OF GOVERNMENT INTEREST

This invention was supported by National Institutes of Health Grant R01 NS33618. The government has certain rights in this invention.

### BACKGROUND OF THE INVENTION

1. Field of the Invention

The present invention is directed to a method for preventing or treating ischemia-reperfusion injuries. In particular, the invention is directed to administering a free radical scavenger to prevent or treat such injuries.

2. Description of the Related Art

Cardiopulmonary bypass utilizing a heart-lung machine enables cardiac surgeons to perform complex cardiac operations including coronary artery bypass grafting, valve repair and replacement, and undertake the correction of congenital heart defects. The technology of cardiopulmonary bypass has improved the survival and quality of life for millions of people worldwide. Unfortunately, despite improvements in blood pumps, oxygenators, filters and other components of the cardiopulmonary bypass circuit, considerable morbidity is associated with the use of this technology.

Cardiopulmonary bypass has been associated with injury to the central nervous system, lungs, kidney, as well as bleeding problems and infections (Cardiopulmonary Bypass, 2^{nd} ed., Gravlee et al., 2000). It has been estimated that at least 3-5% of patients undergoing coronary artery surgery incur serious neurological events such as stroke or transient neurological events such as cognitive decline or cognitive dysfuntion. Cognitive decline complicates early recovery after coronary artery bypass graft (CABG) and may be evident in as many as three quarters of patients at the time of discharge from the hospital and a third of patients after six months (Newman et al., New Engl. J. Med., 344: 395-402, 2001). The rate of cerebrovascular events is even higher in those patients undergoing valvular and combined (valve+CABG) operations. In high-risk patients, the rate of severe postoperative neurological complications has been documented to be as high as 8.4% (Bendszus et al., Arch Neurol. 59: 1090-5, 2002). Neurological complications may be responsible for as many as 20% of the deaths following CABG (Cosgrove et al., Thoracic Cardiovasc. Surg., 88: 673-84, 1984). The majority of these neurologic episodes are likely related to embolic events originating from the cardiac chambers, the aorta, or the carotid arteries (Pugsley et al., Stroke 25: 1393-9,1994; Barbut et al., Ann. Thorac. Surg. 88: 673-84,1997; Kretzschmar et al., Acta Anaesthesiol Scand, 40: 657-64, 1996).

Transient forebrain ischemia secondary to systemic hypotension is common after head injury, cardiac arrest, and shock. About one third of head-injured patients have an episode of significant systemic hypotension. Systemic hypotension may result in neuronal damage to the CA1 region of the hippocampus (See generally, Knuckey et al., Stroke 26: 305-311,1995).

Further, concern regarding the safety of stent implantation in the carotid artery exists because of the risk of cerebral embolization during the procedure and the release of free radicals. A purpose of treating a stenosis of the carotid artery is to prevent stroke. In recent years, stent implantation has been developed as an alternative to surgical treatment for high-grade stenoses in the carotid artery. The distal embolization of plaque material during the procedure is considered the cause of most neurologic complications after stent implantation in the carotid artery (See generally, Jaeger et al., AJNR Am. J. Neuroradiol 23:200-207, 2002). At present, it cannot be completely determined which step of the stent placement procedure causes most of these lesions. Emboli can occur during diagnostic angiography before the intervention. They also may occur during the initial crossing of the stenosis with the guidewire or during balloon angioplasty, stent placement, or post dilation of the stent.

Percutaneous endovascular interventions are rapidly evolving as alternatives to surgical endarterectomy of atherosclerotic stenoses of carotid arteries. Balloon angioplasty effectively resolves stenoses of the carotid arterial bifurcation (See generally, Manninen et al., Analysis Radiology 212: 483-492, 1999). Stents provide an effective means of improving the primary success and long-term patency of peripheral and coronary arteries compared with percutaneous transluminal angioplasty, and show very promising results in atherosclerotic lesions of the carotid arterial bifurcation. Although distal embolism is seldom encountered in peripheral and coronary arteries, it is a feared complication during endovascular procedures in carotid arteries because of its potentially serious consequences.

There is a need for methods that can prevent or reduce ischemia-reperfusion injuries, such as those related to cardiopulmonary bypass, transient forebrain ischemia secondary to systemic hypotension, stent implantation, and percutaneous endovascular intervention. The present invention meets this need, as well as provides other related advantages.

### BRIEF SUMMARY OF THE INVENTION

The present invention provides methods for preventing or treating an ischemia-reperfusion injury. Such methods comprise the step of administering to a subject in need thereof an effective amount of a free radical scavenger prior to, concurrently with, or following reperfusion.

The administration of the free radical scavenger may be intra-arterial, intravenous, intra-peritoneal, oral, intradermal, subcutaneous or transdermal. In certain embodiments, the free radical scavenger is administered intra-arterially (e.g., intra-arterial infusion and administration via the carotid artery) or intravenously. In certain embodiments, the free radical scavenger is delivered to the central nervous system.

As described above, the amount of a free radical scavenger must be sufficient to prevent or treat ischemia-reperfusion injuries. In certain embodiments, depending on the particular free radical scavenger used (e.g., NAC), the dosage may be sufficient for the serum concentration of the free radical scavenger to be from about 1, 2, 3, 4, or 5 mM to about 10, 12, 15, 20, 25, 30, 35, or 40 mM. In certain embodiments, the dosage may be sufficient for the serum concentration of the free radical scavenger to beat least 1, 2, 3, 4, 5, 6, 7, 8, 9, 10, 12, 14, 16, 18, 20, 22, 24, 26, 28, 30, 35, or 40 mM.

The free radical scavenger useful in the present invention may be any compound capable of reducing the amount of free radicals in a cell or tissue. In certain embodiments, the scavenger may comprise NAC, sodium thiosulfate, glutathione ethyl ester, glutathione, D-methionine, cysteamine, cystamine, aminopropylmethylisothiourea, or Ethyol. In certain embodiments, the free radical scavenger is a thiol-containing compound.

The present invention is useful in preventing or treating ischemia-reperfusion injuries. Such injuries include those that occur when the flow of blood to a region of the body is temporarily halted and then re-established. In certain embodiments, the present methods may be used to reduce infarction volume, or treat or prevent cerebral injury such as cerebral hemorrhage and injury associated with a cardiopulmonary bypass procedure (e.g., CABG).

### BRIEF DESCRIPTION OF THE SEVERAL VIEWS OF THE DRAWINGS

FIG. 1a shows ischemic infarction 24 hours after 60 minute middle cerebral artery occlusion (MCAO) in the rat demonstrated on 2,3,5-triphenyltetrazolium chloride (TTC) stained brain slices taken 6 mm distance from the frontal pole, the major distribution area of the middle cerebral artery (MCA).

FIG. 1b shows ischemic infarction 24 hours after 60 minute MCAO demonstrated on TTC stained brain slices taken 8 mm distance from the frontal pole, the major distribution area of the MCA.

FIG. 2 shows a graph representing the effect of NAC (400 mg/kg i.v.) pretreatment on infarction volume 24 h after 60 min MCAO.

FIG. 3 shows experimental series and groups: timing of NAC, saline and L-buthionine-[S,R]-sulfoxamine (BSO) administration. In series A, Group 1 (NAC) and Group 2 (saline) animals were pretreated for 60 min prior to occlusion, and in series B, Group 3 (NAC) and Group 4 (saline) were pretreated for 30 min prior to occlusion. In series C animals were treated with NAC (Group 5) or saline (Group 6) 2 minutes after reperfusion. In Series D the animals were treated with BSO twice daily for 3 days. Then animals were pretreated for 60 min with NAG (Group 7) or saline (Group 8) prior to occlusion, as in series A. Animals in series E (Group 9) underwent middle cerebral artery occlusion without any treatment.

FIGS. 4A and 4B show TTC staining of coronal brain sections (2 mm) 24 h after reperfusion following 1 h middle cerebral artery occlusion in representative saline (A) and NAC pretreated animals (B). NAC or saline was administered 60 min prior to occlusion. Unstained areas show infarction. FIGS. 4C and 4D show H&E stained paraffin sections from a representative stoke animal. Whole mount shows edema and vacuolation of the left hemisphere cortex and the majority of the striatum (C). High power (20× objective) of entorhinal cortex shows classic cytologic ischemic changes of 24 hour duration, neuronal pyknosis, loss of Nissl substance and edema (D).

FIG. 5 shows infarction areas measured on individual TTC stained coronal brain sections (2 mm) 24 h after reperfusion following 1 h middle cerebral artery occlusion in three different experimental settings. The graphs show infarction area in percentage of the affected hemisphere, mean±SD. Series A animals were pretreated with saline or NAC at 60 min prior to occlusion, as series B animals at 30 min prior to occlusion. In series C animals received NAC or saline 2 minutes after reperfusion.

FIG. 6 shows calculated total infarction volume in different experimental series measured on 2,3,5-triphenyltetrazolium chloride stained coronal brain sections (2 mm) 24 h after reperfusion following 1 h middle cerebral artery occlusion. The graph shows meant±SD in mm³. In series A, animals were pretreated with saline or NAC at 60 min prior to occlusion, as series B animals at 30 min prior to occlusion. In series C, animals received NAC or saline 2 minutes after reperfusion. The last column displays infarction measured in untreated control stroke animals. Significant reduction of total infarction volume was observed in NAC versus saline treated animals in series A and B (p<0.05).

### DETAILED DESCRIPTION OF THE INVENTION

The present invention provides methods for preventing or treating ischemia-reperfusion injuries. Such methods comprise administering to a subject in need thereof an effective amount of a free radical scavenger, such as NAC.

As used herein, “ischemia-reperfusion injury” refers to any injury occurring when the flow of blood to a region of the body is temporarily halted (ischemia) and then re-established (reperfusion). Ischemia-reperfusion injury can occur during certain surgical procedures, such as organ injury during organ transplantation; brain injury during carotid artery surgery, cerebral vascular surgery and surgery of the heart and aorta; brain, spinal cord, intestine and kidney injury during surgery of the thoracic aorta and kidney injury during abdominal aortic surgery; injury to the central nervous system, lungs, kidneys following thromboembolectomy or the use of cardiopulmonary bypass during lung and heart surgery; heart injury following revascularization (coronary artery bypass graft surgery); kidney injury following surgery on renal arteries; intestinal injury following surgery on the mesenteric arteries; and skin injury following harvesting of a skin graft. Ischemia-reperfusion injury may also occur during angioplasty or thrombolytic therapy, including stent implantation and percutaneous endovascular interventions. Further, ischemia-reperfusion injury may be induced by injuries or conditions such as bowel ischemia and perfusion, sepsis, anaphylaxis, hemorrhagic shock and trauma, systemic hypotension, embolisms and infarctions.

Clinically ischemia-reperfusion injury may be manifested by such complications as cerebral infarction, cognitive dysfunction, pulmonary dysfunction (e.g., adult respiratory distress syndrome), renal dysfunction, consumptive coagulopathies (e.g., thrombocytopenia), fibrin deposition into the microvasculature and disseminated intravascular coagulopathy, transient and permanent spinal cord injury, cardiac arrhythmias and acute ischemic events, hepatic dysfunction (e.g., acute hepatocellular damage and necrosis), gastrointestinal dysfunction (e.g., hemorrhage and/or infarction), and multisystem organ dysfunction (MSOD) or acute systemic inflammatory distress syndromes (SIRS). The injury may occur in the parts of the body to which the blood supply was interrupted, or it can occur in parts fully supplied with blood during the period of ischemia. In the affected tissues, neutrophil infiltration, hemorrhage, edema and necrosis are frequently observed.

“Preventing an ischemia-reperfusion injury” refers to preventing or diminishing the occurrence of an ischemia-reperfusion injury. A subject in need of prevention of ischemia-reperfusion injuries refers to a human, non-human primate or other animal that is at risk for ischemia-reperfusion injuries. It includes an animal that will undergo, or is undergoing, a clinical procedure that may induce ischemia-reperfusion injuries, and an animal with injuries or conditions likely to cause ischemia-reperfusion injuries.

“Treating an ischemia-reperfusion injury” refers to ameliorating an ischemia-reperfusion injury. It includes ameliorating the injury of affected tissues or organs, increasing the survival of affected cells, decreasing infarction volume, and the like. A subject in need of treatment of ischemia-reperfusion injuries refers to an animal (e.g., human) with an ischemia-reperfusion injury.

The term “effective amount” refers to a concentration of a free radical scavenger that is sufficient to prevent or reduce an ischemia-reperfusion injury.

“Free radical scavenger” refers to a compound capable of reducing the amount of free radicals in a cell or tissue. It includes, but is not limited to, NAC, sodium thiosulfate, glutathione ethyl ester, glutathione, D-methionine, cysteamine, cystamine, aminopropylmethylisothiourea, Ethyol, vitamin E, Edaravone (3-methyl-1-phenyl-2-pyrazolin-5-one), melatonin, polynitroxyl-albumin, idebenone, nitric oxide, Carvedilol, alpha-lipoic acid, allopurinol, 2-O-octadecylascorbic acid and N-2-mercaptopropionyl glycine (see, e.g., Tang and Tang, Yao Xue Xue Bao 26: 91-5, 1991; Boaz et al., Lancet 356: 1213-8, 2000; Tanaka, Nippon Yakurigaku Zasshi 119: 301-8, 2002; Tan et al., Curr. Top. Med. Chem. 2: 181-97, 2002; Martin et al., Eur. J. Cardiothorac. Surg. 19: 321-5, 2001; Grieb et al., Resuscitation 39: 107-13,1998; Fukahara et al., Eur. J. Cardiothora. Surg. 11: 343-9, 1997; Ma et al., J. Pharmacol. Exp. Ther. 277:128-36, 1996; Free Radic. Res. 23: 365-70, 1995; Oredsson et al., Eur. J. Vasc. Surg. 5: 47-52, 1991; Tara et al., J. Cardiovasc. Pharmacol. 16: 984-91, 1990; Mitsos et al., J. Cardiovas. Pharmacol. 8: 978-88,1986). Preferably, the free radical scavenger is a thiol-containing compound (e.g., NAC). Free radical scavengers of the present invention may be used individually or in combination with one or more other free radical scavengers, or other pharmaceutical agents and excipients.

Free radicals are natural but generally undesirable byproducts of cell metabolic processes in different subcellular compartments and membranes. These radicals (e.g., superoxide ion, hydroxyl radicals, nitric oxide) are highly reactive and destructive to cells and/or tissues because of the presence of unpaired electrons. In normal systems, injury from these radicals is prevented or minimized by radical scavenging systems. However, when blood is reperfused to an area previously exposed to ischemia, free radicals reportedly are formed at a greater rate than they can be scavenged by natural radical scavenging systems (Lyrer et al., Brain Res. 576: 317-20, 1991). In addition, ischemia-reperfusion has also been reported to cause a decrease in the levels of free radical scavengers (Landolt et al., Brain Res. 567:317-20, 1991). For instance, elevated oxygen levels following the reperfusion cannot be utilized by the mitochondria that have been damaged by ischemia, providing more oxygen for enzymatic oxidation and production of free radicals.

Without being bound to theory, it is thought that the free radical scavengers according to the present invention (e.g., NAC) prevent or reduce ischemia-reperfusion injuries by reducing the amount of free radicals in cells and tissues. For example, in vitro experiments have demonstrated that NAC scavenges the hydroxyl radical that is generated after forebrain ischemia (see generally, Knuckey et al., Stroke 26:305-311, 1995). NAC also has indirect free radical scavenging potential because NAC is deacetylated to cysteine, a thiol reducing agent, which supports glutathione biosynthesis. Cysteine is the limiting substrate of glutathione biosynthesis and may easily cross the blood brain barrier (BBB) via specific amino acid transporter. In patients 30 minutes after the systemic administration of NAC (150 mg/kg), increased GSH and cysteine levels were detected in the plasma. Glutathione may cross the BBB via a carrier-mediated mechanism (Kannan et al., J. Pharmacol. Exp. Ther. 263: 964-70, 1992) and exert a direct free radical scavenging effect. Glutathione levels are reportedly depleted after transient focal ischemia (Lyrer at al., Brain Res. 576: 317-20, 1991) and traumatic brain injury (Xiong et al., J. Neurotrauma 16: 1067-82, 1999). Administration of NAC restored glutathionc levels after traumatic brain injury (Xiong et al., J. Neurotrauma 16: 1067-82, 1999). The effect of NAC in the restoration of glutathione levels may contribute to the neuroprotective mechanism in ischemic brain injury as well.

While the scavenging of free radicals is the most likely mechanism of action of free radical scavengers, there are several other possibilities that may account for desirable effects of certain scavengers on ischemia-reperfusion injury. For instance, ischemic-reperfusion injury has a deleterious effect on the microvascular and endothelial function that may be ameliorated by some scavengers, such as NAC. The disturbance of endothelial cells and the accumulation of neutrophils after ischemia result in stimulation of nitric oxide synthase and generation of nitric oxide. The nitric oxide reacts with the superoxide ion, with the production of peroxynitrate. The increased production of nitric oxide appears to be detrimental to neuronal survival because inhibition of nitric oxide synthase decreases cortical infarction (Kuluz et al., Stroke 24: 2023-2029,1993). The antioxidant NAC inhibits nitric oxide formation, which is thought to be due to the inhibition on nitric oxide synthase (Cuzzocrea at al., Br. J. Pharmacology 13: 1219-26, 2000). In addition, studies also suggest that NAC may improve microcirculatory blood flow and tissue oxygenation (Cuzzocrea at al., Br. J. Pharmacol. 13: 1219-26, 2000). NAC was reported to enhance oxygen extraction in patients with culminant liver failure (Harrison et al., N. Eng. J. Med. 324: 1852-7, 1991). It was further shown to promote survival of sympathetic neurons in the absence of trophic factors via the activation of the Ras-extracellular signal-regulated kinase (ERK) (Yan and Greene, J. Neusci. 18: 4042-9, 1998).

The free radical scavengers of the present invention are formulated to be compatible with their intended route of administration. Examples of route of administration include intravenous (i.v.), intra-arterial (i.a.), intra-peritoneal (i.p.), oral (p.o.), intradermal, subcutaneous, and transdermal administration. Solutions or suspensions used for intravenous, intra-arterial, intradermal, or subcutaneous application can include one or more of the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. In addition, pH may be adjusted with acids or bases, such as hydrochloric acid or sodium hydroxide. The free radical scavengers are preferably administered in their un-oxidized form. The parenteral preparation can be enclosed in ampoules, disposable syringes or multiple dose vials made of glass or plastic.

Free radical scavengers suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against contamination from microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

It may be desirable to deliver free radical scavengers to the central nervous system (CNS) in certain embodiments (e.g., preventing or treating cerebral ischemia-reperfusion injury). Various methods for delivering chemical compounds to the CNS are known in the art and may be used in the present invention. These methods include, but are not limited to, carrier-mediated transporters (see, e.g., Pardridge, in Brain Drug Targeting: The Future of Brain Drug Development, pp 1-346, Cambridge University Press, Cambridge, 2001), active efflux transporters, such as p-glycoprotein (see, e.g., Tsuji, Ther. Drug Monit. 20: 588-90, 1998; Jolliet-Riant and Tillement, Fundam. Clin. Pharmacol. 13: 16-26,1999), nanoparticulate systems (see, e.g., Kreuter, Adv. Drug Deliv. Rev. 47: 65-81, 2001), polymeric delivery device (see, e.g., U.S. Pat. No. 5,601,835), and transient opening of the BBB by intracarotid infusion of hypertonic mannitol solutions or of bradykinin analogs (Jolliet-Riant and Tillement, Fundam. Clin. Pharmacol. 13: 16-26,1999; Neuwelt et al., Cancer Res. 61: 7868-74, 2001).

In certain embodiments, free radical scavengers (e.g., NAC) are preferably administered i.v. or i.a. Such routes of administration provide a biodistribution of the scavengers different from i.p. or p.o. Oral administration is analogous to intra-peritoneal because most intra-peritoneal drugs are absorbed by intestine. The venous drainage for the gastrointestinal tract is the portal vein, which clears most drugs on the first pass uptake by the liver. Intravenous drugs are less cleared on first pass uptake by the liver, and intra-arterial drugs go to tissues (e.g., brain) before liver. Although certain free radical scavengers (e.g., NAC) only minimally cross the BBB when given i.v. (McLellan et al., Carcinogenesis (Lond.) 16: 2099-106,1995), they can cross the BBB when given i.a. into the carotid artery with or without osmotic BBB disruption (Neuwelt et al., Cancer Res. 61: 7868-74, 2001).

In addition, appropriate administration routes may be selected depending on the target tissue or organ to which free radical scavengers need to be delivered. For instance, the aortic infusion route is preferred for prevention of bone marrow toxicity of free radical scavengers (e.g., NAC). Aortic infusion avoids the first pass clearance in the liver and kidneys, and provides maximal delivery to the target (i.e., bone marrow). This route of delivery may not be optimal for prevention of toxicity of free radical scavengers (e.g., NAC), such as brain or liver, in which intravenous delivery may provide higher first pass concentration.

It is advantageous to formulate compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. While compounds that exhibit toxic side effects may be used, care should be taken to design a delivery system that targets such compounds to the site of affected tissue to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Various cell culture assays and animal models for evaluating effectiveness of a particular free scavenger in preventing or treating ischemia-reperfusion injuries are known in the art. They include, but are not limited to those described in U.S. Pat. Nos. 6,086,868; 6,001,842; 5,968,959; 5,912,019; 5,869,044; and 5,648,331; Sekhon et al., Brain Res. 971: 108, 2003; Tripathi and Hegde, Indian J. Physiol. Pharmacol. 42: 50-6, 1998; Horowitz, Am. J. Med. 91: 113S-117S, 1991; Koksal et al., J. Surg. Res. 111: 236-9, 2003; Sener et al., Life Sci. 72: 2707-18, 2003; Sehirli et al., J. Nephrol. 16: 75-80, 2003; Weinbroum et al., Med. Sci. Monit. 7: 1137-44, 2001; Weinbroum et al., Transplantation 27: 300-6, 2001; Weinbroum et al., Transplantation 69: 853-9, 2000; Borjesson et al., Dig. Surg. 17: 379-87, 2000; Cuzzocrea et al., Cardiovasc. Res. 47: 537-48, 2000; Currocrea et al., Br. J. Pharmacol. 130: 1219-26, 2000; Chavez-Cartaya et al., Transpl. Int. 12213-21, 1999; Silbergleit et al., Resuscitation 40: 181-6, 1999; Salom et al., Transplantation 65: 1315-21, 1998; DiMari et al., Am. J. Physiol. 272: F292-8, 1997; Kretzschmar et al., Acta Anaesthesiol Scand. 40: 657-64, 1996; Koeppel et al., Eur. Surg. Res. 28: 270-7, 1996; Nakano et al., Eur. Surg. Res. 28: 245-55, 1996).

Certain free radical scavengers have been clinically used for treatments other than preventing or reducing ischemia-reperfusion injuries. For instance, NAC has been used clinically as a mucolytic agent and has been approved by the US Food and Drug Administration (FDA) for acetaminophen poisoning. The oral dose approved by the FDA for treatment of acetaminophen poisoning is 140 mg/kg.

The dosage of using NAC in preventing or treating ischemia-reperfusion injuries may be at least 150, 200, 250, 300, 350, or 400 mg/kg to at most 800, 850, 900, 950, 1000, 1100, 1200, 1300, 1400, 1500, or about 1600 mg/kg in rats, or a dosage in another subject comparable to that in rats. A dosage (“dosage X”) of a free radical scavenger in a subject other than a rat is comparable to a dosage (“dosage Y”) of the free radical scavenger in rats if the serum concentration of the scavenger in the subject post administration of the scavenger at dosage X is equal to the serum concentration of the scavenger in rats post administration of the scavenger at dosage Y. Previous published work has used a dose of approximately 140 mg/kg, the human dose for acetaminophen toxicity. Multiple low doses are required to see protective activity against ischemia-reperfusion injuries. It was shown by the present inventors that in rats, at this low dose (i.e., about 140 mg/kg), serum NAC concentrations (5 min post infusion) were in the range of 0.1-0.3 mM; whereas when administrated at 400 mg/kg or 1000 mg/kg, serum NAC concentrations were about 1.5 mM and 10 mM, respectively. In addition, a single high dose of NAC (400-1000 mg/kg) was surprisingly found to be more effective in preventing or treating ischemia-reperfusion injuries than multiple low doses of NAC. Thus, in certain preferred embodiments, the dosage for using NAC in preventing or treating ischemia-reperfusion injuries is sufficient to obtain a serum concentration in a subject in need thereof equal to, or higher than, the serum NAC concentration that would be reached by the administration of NAC at a dosage at least 400, 500, 600, 700, 800, 900, 1000, 1100, or 1200 mg/kg in rats. For example, in certain embodiments, the serum NAC concentration in a subject (e.g., a human) shortly post administration (e.g., 5 minutes post infusion) is at least 1, 2, 3, 4, 5, 6, 7, 8, 9, 10, 12, 14, 16, 18, 20, 22, 24, 26, 28, 30, 35, or 40 mM. In addition, multiple high dose regimens may also be used. For instance, NAC (as well as other free radical scavengers) may be administered at least 2, 3, 4, 5, 6, 7, 8, 9, or 10 times at one of the above dosages every 1, 2, 3, 4, 5, 6, 7, 8, 9, 10, 11, or 12 hours. No toxicity was observed in rats treated with 1000 mg/kg NAC intra-arterially or intravenously every 8 h three times.

Free radical scavengers may be administered to a subject in need thereof prior to, concurrent with, or following ischemia-reperfusion injuries. For instance, free radical scavengers may be administered to a subject at least 2 hours, 1.5 hours, 1 hour, or 30 minutes before potential ischmeia-reperfusion injuries such as those associated with certain surgical procedures. In certain embodiments, they may be administered concurrent with or following such surgical procedures. In other embodiments, free radical scavengers may be administered following the detection of ischemia-reperfusion injuries. Generally, these scavengers are administered for a sufficient period of time so that ischemia-reperfusion injuries are prevented or treated.

In certain embodiments, an appropriate dosage of a free radical scavenger (e.g., NAC) is combined with a specific timing and/or a particular route to achieve the optimum effect of a free radical scavenger in preventing or treating ischemia-reperfusion injuries. For instance, NAC may be administered to a subject at a dosage sufficient to obtain serum NAC concentration at least 1, 2, 3, 4, 5, 6, 7, 8, 9, 10, 12, 14, 16, 18, 20, 22, 24, 26, 28, 30, 35, or 40 mM, via i.v. or i.a., at least 30 minutes, 1 hour, 1.5 hours or 2 hours before ischemia-reperfusion injuries.

As described above, the present invention provides methods for preventing or treating ischemia-reperfusion injuries comprising administering to a subject in need thereof an effective amount of free radical scavengers. In certain embodiments, the present methods are particularly useful in preventing or treating cerebral ischemia-reperfusion injuries such as infarction, breakdown of BBB, and cerebral hemorrhage. Cerebral ischemia-reperfusion injuries may be associated with certain clinical procedures such as the use of a heart-lung machine (pump) during cardiopulmonary bypass, stent implantation, open heart surgery, certain neurosurgical procedures requiring temporary arterial occlusion, and percutaneous endovascular interventions. In addition, such injuries may also be associated with medical conditions, for example, systemic hypotension. The neuroprotective effect of free radical scavengers may be evaluated using various techniques, in vitro cultured cell systems, and/or animal models known in the art (see, e.g., Silbergleit et al., Resuscitation 40: 181-6,1999; Hori et al., Brain Res. 652: 304-10,1994; Tian et al., Neurosci. Res. 47: 47-53, 2003; Sekhon et al., Brain Res. 971: 1-8, 2003; Cuzzocrea et al., Br. J. Pharmacol. 130: 1219-26, 2000; Carroll et al., Brain Res. Mol. Brain Res. 56:186-91, 1998).

For instance, the present invention provides methods for preventing or treating cerebral ischemia-reperfusion injuries associated with the use of cardiopulmonary bypass circuit. Such methods comprise the step of administering an effective amount of a free radical scavenger to a subject prior to, concurrently with, or following a surgery involving the use of cardiopulmonary bypass circuit (e.g., a heart-lung machine). The cerebral injuries associated with the use of cardiopulmonary bypass circuit include, but are not limited to, stroke, focal injury, cognitive dysfunction, deterioration in intellectual function, memory deficit and seizures (Roach et al., N. Eng. J. Med. 335: 1857-63, 1996). The effectiveness of the treatment may be monitored and evaluated by appropriate methods known in the art, such as CT scan, MRI, transcranial doppler ultrasonography, auditory evoked potentials, release of S-100 and neurocognative testing (see, e.g., Barbut et al., Ann. Thorac. Surg. 64: 454-9,1997; Andersen et al., Perfusion 10: 21-6, 1995).

The present invention also provides methods for reducing ischemia-reperfusion induced cerebral hemorrhage. Such methods comprise the step of administering a free radical scavenger to a subject in need thereof in an amount sufficient to reduce cerebral hemorrhage. Ischemic injuries include localized tissue anemia due to obstruction of the inflow of arterial blood. Ischemic injuries to the central nervous system result in BBB breakdown, allowing increased transit of fluid and plasma constituents into the brain parenchyma. Ischemic opening of the BBB after reperfusion is a major contributor in the development of brain edema and hemorrhagic transformation in ischemic areas. Previous studies suggest differential opening of the BBB for low and high molecular weight particles in ischemic stroke (Preston et al., Acta Neuropathol. 103: 237-242, 2002; Preston et al., Brain Research 761:4-10 (1997). A free radical scavenger reduces cerebral hemorrhage if the amount of cerebral hemorrhage is statistically significantly less in the presence of the scavenger than in the absence of the scavenger. An appropriate free radical scavenger may be selected and the effectiveness of the treatment may be evaluated using methods and/or animal models known in the art, such as CT scan, ^{99m}Tc-hexamethylpropylenamine oxime single-photon emission computed tomography (SPECT) scan, and MRI (see, e.g., Mayer et al., Stroke 29: 1791-98,1998; Knight et al., Stroke 29: 144-51,1998; Zhao et al., Stroke 32: 2157-63, 2001; Dijkhuizen et al., J. Cereb. Blood Flow Metab. 21: 964-71, 2001; Tejima et al., Stroke 32: 1336-40, 2001; Neumann-Haefelin et al., Neuroreport 12: 309-11, 2001; McCabe et al., Stroke 30: 2483-6,1999; Fagan and Garcia, Pharmacotherapy 19: 139-42,1999; Hamann et al., J. Cereb. Blood Flow Metab. 16: 1373-8,1996; Masdeu and Brass, J. Neuroimaging Suppl. 1: S14-22,1995; Broderick et al., Stroke 26: 484-7, 1995).

The following examples are provided to illustrate the invention and are not to be construed as a limitation thereon.

### EXAMPLE 1

### Neuroprotection Experiments

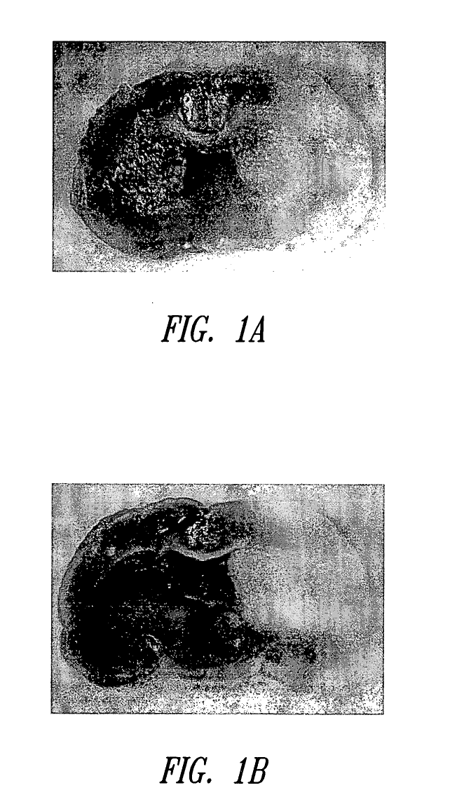

Twenty female Long Evans rats underwent 60 minute transient MCAO. The rats were anesthetized, intubated and mechanically ventilated using 2% Isoflurane, available from Abbott Laboratories, North Chicago, Ill., and oxygen mixture. The left common carotid artery (CCA) and the bifurcation were exposed via midline incision. The branches of the left external carotid artery (ECA) were coagulated, and the ECA stump was mobilized inferiorly, the left pterygopalatine artery was ligated. A blunt tipped 3 cm long segment of a 3-0 monofilament nylon suture, available from Dermalone, United States Surgical, Norwalk, Conn., was introduced into the left internal carotid artery via the left ECA stump, and advanced 2.0-2.2 cm distance from the bifurcation. Then the proximal CCA was ligated temporarily. After the defined occlusion time the animal was re-anesthetized using a mask. The ligation of the CCA was released, and the suture was pulled back and removed allowing reperfusion of the occluded segment. Animals received either saline or 400 mg/kg of NAC, available from Abbott Laboratories, North Chicago, Ill., in 2.5 ml saline via the left femoral vein over 15 minutes, starting 30 min before the occlusion. All animals showed clinical signs of stroke. The animals were sacrificed 24 h later with intracardiac injection of Pentobarbital. As shown in FIGS. 1a and 1b, the brains were removed, sliced in 2 mm thick sections, which were stained in 2% TTC solution for 15 minutes at 37° C. The animals having significant subarachnoid hemorrhage, or animals having no strokes with TTC staining were excluded from the study (n=6). Seven animals from both the NAC and the control group were eligible for the further investigation. The slices were photographed through the operating microscope using a Kodak DX3600 2.2 Mpx digital camera. The pictures were analyzed using the Scion Image for Windows software (based on NIH Image Software for Macintosh). The non-stained area was outlined, and the area was calculated, as a percentage of the stroked hemisphere. Data were analyzed and graphed using Microsoft Excel Software.

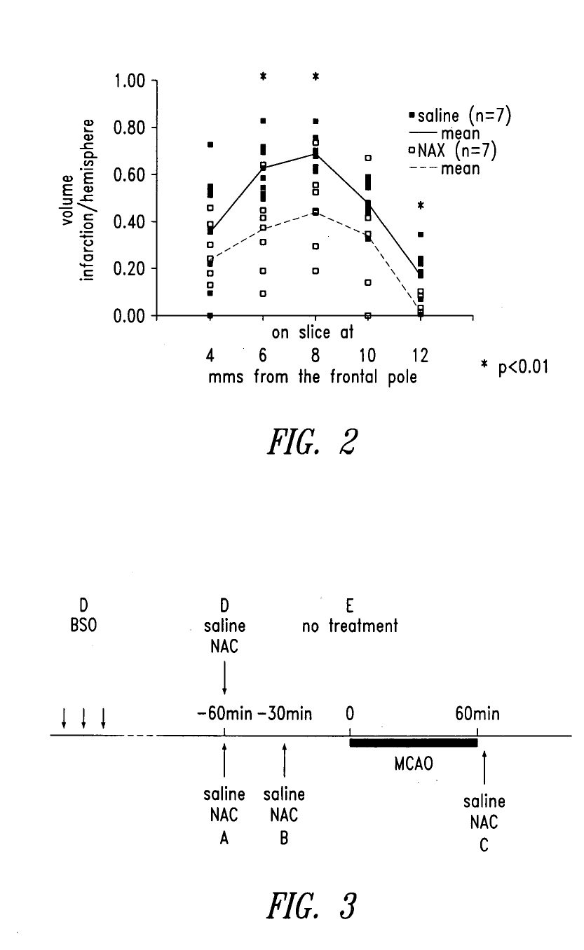

All animals included in the NAC study developed stroke in the left hemisphere. The infarction was identified as low or non-staining area with TTC. As shown in FIG. 2, digital image analysis showed that the infarcted area was smaller by a mean of 38% in the NAC pretreated animals on the slices taken 6 and 8 mms from the frontal pole, which represents the major distribution area of MCA, than in the control group. The difference was significant on two-sided t test (p<0.01). The high standard deviation in the initial slices is due to the inconsistent occlusion of the anterior cerebral artery, which depends on the position of the tip of the intraluminal suture and the individual anatomy. In slices taken 10 and 12 mms from the frontal pole, the infarction was smaller in the NAC group, but a significant difference was observed only in the last slice.

### EXAMPLE 2

### Beneficial Effect of N-Acetylcysteine Prophylaxis on Cerebral Infarction Volume in a Rat Model of Focal Transient Ischemia and Reperfusion

Surgical Preparations

Temporary (60 vain) middle cerebral artery occlusion was performed in female Long-Evans rats weighting 220-270 grams using modified intraluminal suture technique. (Longa et al., Stroke 20: 84-91,1989; Aspey et al., Neuropath. Appl. Neurobiol. 24: 487-97,1998; Aspey et al., Neuropath. Appl. Neurobiol. 26: 232-42, 2000). Anesthesia was induced with 5% Isoflurane (Abbot Laboratories, North Chicago, Ill.). Once the animal was sedated, the animal was intubated (16G Cathlon catheter, Johnson & Jonhson Medical, Arlington, Tex.) and mechanically ventilated with 2% Isofturane (Harvard Animal Ventilator, Model 683, Holliston, Mass.). The left carotid bifurcation was exposed via a median skin incision with retraction of the left sternocleidomastoid muscle and transection of the left omohyoid muscle under an operating microscope. The proximal branches of the left external carotid artery were coagulated and transected. The ECA was ligated and transected above the origin of the superior thyroid artery, and the stump was mobilized caudally. Then the bifurcation of the internal carotid artery was exposed and the origin of the ptcrygopalatineartery was ligated. After proximal clipping, the ECA stump was opened and a 3 cm long, blunt tipped 3-0 monofilament Nylon suture (Dermalon, USS, Norwalk Conn.) was introduced into the ICA and gently advanced into the ICA about 2.0-2.2 cm distance from the bifurcation, until resistance was felt. Then the intraluminal suture was secured with a ligature of the ECA, and temporary ligature was applied to the CCA as well. The wound was closed and the animal was allowed to wake up and was placed in an incubator. Sixty minutes after the occlusion, if the animal showed obvious signs of left hemisphere ischema (right forelimb flexion, aversion), the animal was re-anesthetized using a facemask. The incision was opened, the ligature around the CCA was released to allow reperfusion of the ICA and the suture was gently drawn back and removed. The ECA stump was then coagulated and the wound was closed. Body temperature was maintained using heating pad (Harvard Homeothermic Blaket Control Unit, Holliston, Mass.) throughout the experiment.

Experimental Protocols

Previous studies (Neuwelt et al., Cancer Res. 61: 7868-74, 2001) reported increased toxicity of intravenous versus intraarterial administration of NAC. A pilot study using escalating intravenous dose of NAC showed that intravenous administration of 400 mg of NAC in 5 min infusion did not cause significant morbidity or mortality in stroke animals. Three animals underwent physiological monitoring during and after intravenous administration of NAC and except of transient decrease (10%) of mean arterial blood pressure, no other significant changes were observed in vital parameters.

The possible neuroprotective effects of NAC in four experimental series were investigated (FIG. 1), each containing animals randomized to NAC or saline treatment groups. NAC (Acetyleystcine, Abbot Laboratories, North Chicago, Ill.) 400 mg/kg in 2500 μl volume or saline (0.9% sodium chloride solution, Abbot Laboratories, North Chicago, Ill.) was infused at a rate of 500 μl/min for 5 min via the left femoral vein under isoflurane anesthesia. In series A, Group 1 (saline) and Group 2 (NAC) animals were pretreated for 60 min prior to occlusion, and in series B, Group 3 (saline) and Group 4 (NAC) were pretreated for 30 min prior to occlusion. In series C, animals were treated with saline (Group 5) or NAC (Group 6) 2 minutes after reperfusion. In series D, the effect of NAC on glutathione-depleted animals was investigated to test the involvement of the glulathione scavenger system. For GSH depletion, the animals were treated with 10 g/m² of L-buthionine-[S,R]-sulfoxamine (BSO, supplied by the National Cancer Institute, NIH, Bethesda., M15) administered intraperitoneally twice daily for 3 days. This regimen causes at least 50% reduction of brain glutathionc levels as previously reported (Neuwelt et al., Cancer Res. 61: 7868-74, 2001). The animals were pretreated for 60 min with saline (Group 7) or NAC (Group 8) prior to occlusion, as in series A. In Group E for untreated controls, 6 animals underwent 60 min MCAO without any treatment. See Table 1 for treatment groups.

Measurement of Infarction Volume

Twenty four hours after surgery the animals were anesthetized with 5% Isollurane, and the animals were sacrificed using 1 cc intracardiac injection of Pentobarbital (Nembutal, Abbot, Chicago, Ill.). The brains were quickly removed, rinsed, and sliced into 2 man thick sections starting at the frontal pole in a standard rat brain matrix (Harvard Apparatus, Holliston, Mass.). The brain slices were then stained for 15 minutes at 37° C. in 2% TTC solution (Sigma, St. Louis, Mo.). TTC immersion staining has been demonstrated to provide adequate assessment of ischemic area in the first 24 hours of infarction (Hatfield et al., Neuropathol. Appl. Neurobiol. 17: 61-7,1991; Park et al., Neuropathol. Appl. Neurobiol. 14: 289-98, 1988). The infarction was visible as areas with loss of the red TTC staining. The slices were then immersion fixed in buffered 4% formaldehyde solutions. Caudal surface of five slices (slices #2-6) per animal were analyzed using the Scion Image Beta 4.02 for Windows Acquisition and Analysis Software (Scion Corporation, 2000, Frederick, Md.). The non-stained area was outlined and the area was measured, and calculated as percentage of the affected hemisphere. Total lesion volume was calculated by summing the infarction volume measured of each section. The infarction volume in one slice was calculated by multiplying slice thickness and the average of the infarction area measured on both sides of the slice.

Statistical Analysis

Mixed model repeated measures analysis of variance models (Littell et al., 1996) were fit to the data to determine differences among series and groups within series for infarction ratios and areas. Compound symmetry and autoregressive covariance structures were each fit to these data, and the covariance structure more appropriate to the data was selected using Kike's information criterion (Littell et al., 1996). Least square means (95% confidence intervals) were computed and compared using Tukey-Kramer approximation to adjust for multiple comparisons. For comparisons of saline and NAC groups, separate analyses are performed for each series (i.e., A, B and C). To account for multiple comparisons, a Bonferroni adjustment was used for each series specific analysis. A one-way analysis of variance was computed to determine difference in total infarction volume among 7 groups. These groups are NAC and saline for each of series A, B, and C and for the no treatment group. Means were compared pairwise for these groups to determine if there were significant differences with a Tukey-Kramer adjustment applied for multiple comparisons. Due to the low animal count, a separate analysis was performed to compare total infarction volumes in series D using the Student's t-test. Differences were considered significant if p<0.05. All analyses were run using SAS® Version 8 for Windows (SAS Institute, Cary, N.Y., 1999-2001). All data displayed as unadjusted mean±standard deviation. Data was stored and graphed using Microsoft Excel® software.

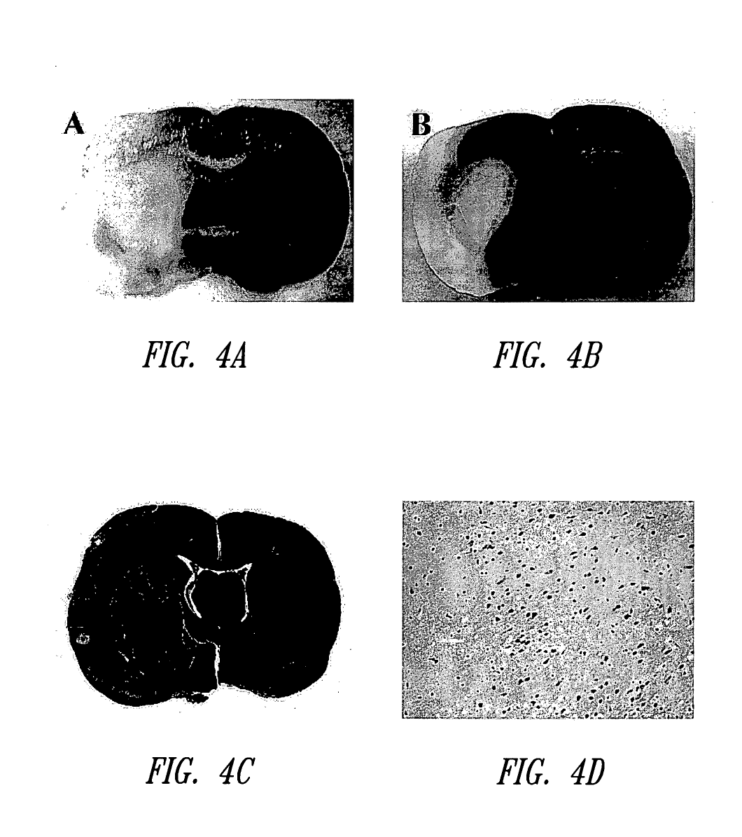

Animals not showing clinical signs of left hemispheric ischemia (right forelimb flexion and circling) and presenting with no infarction with TTC staining or showing evidence of subarachnoid hemorrhage were excluded from the study. 66 animals of 90 total were eligible for further investigations. FIG. 4 demonstrates representative slices stained with TTC in animals with and without NAC treatment. Neuronal loss is shown on HE stained slices in FIG. 4C. The total infarction volume in the different experimental settings is displayed in Table 1.

TABLE 1
Experimental series, treatment groups, timing of N-acetylcysteine
(NAC) or saline administration, and measured total infarction
volumes after 60 min occlusion and 24 h reperfusion.

| | | | | | infarction |
|---|---|---|---|---|---|
| | | | | | volume |
| Series | Group | n | treatment | timing | (mm3) |
| A | 1 | 10 | saline | −60 min to | 249.54 ± 55 |
| | 2 | 12 | NAC | MCAO | 143.40 ± 72* |
| B | 3 | 10 | saline | −30 min to | 266.82 ± 41 |
| | 4 | 11 | NAC | MCAO | 176.60 ± 69 |
| C | 5 | 6 | saline | reperfusion | 200.34 ± 33 |
| | 6 | 5 | NAC | | 156.41 ± 27 |
| D | 7 | 3 (4†) | BSO + | −60 min to | 239.38 ± 58 |
| | | | saline | | |
| | 8 | 3 | BSO + | MCAO | 218.14 ± 74 |
| | | | saline | | |
| E | 9 | 6 | Untreated | | 249.08 ± 30 |

Values are displayed as mean ± SD.
†One animal died due to ischemic edema and hemiation, no volumetrics obtained.
*Significant reduction of total infarction volume was observed in NAC versus saline heated animals on series A and B (p < 0.05).

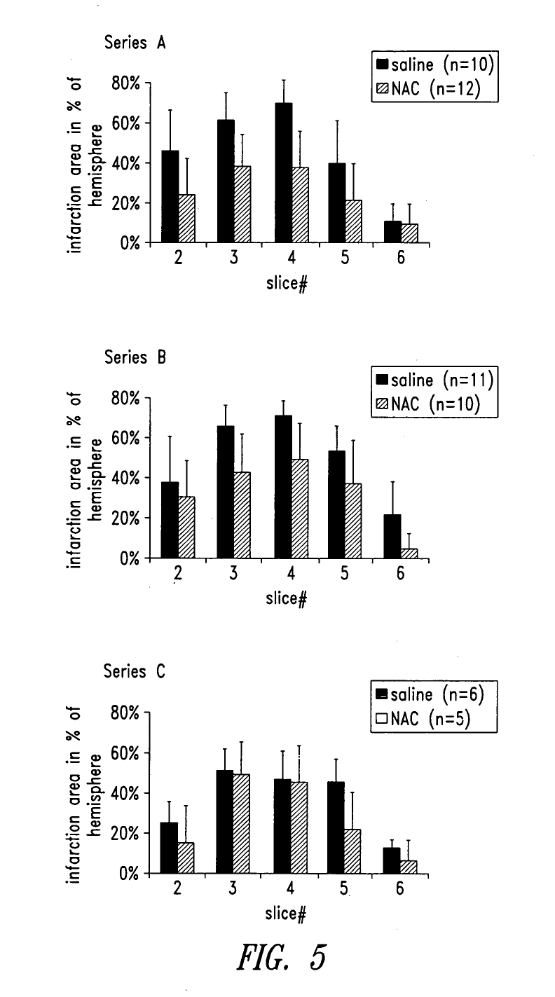

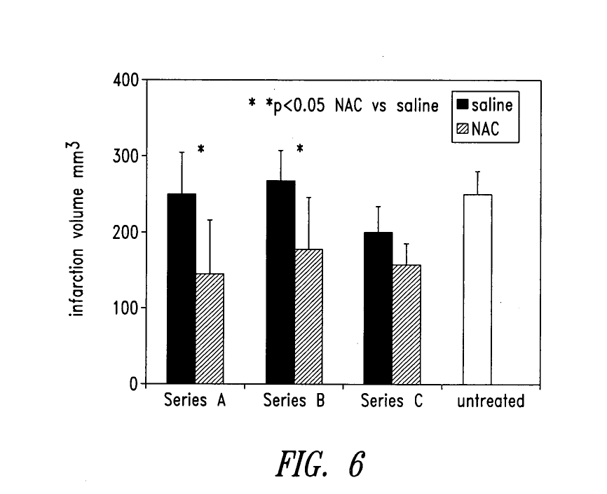

The difference among the means of the 7 groups was statistically significant (p<0001). There were no significant differences comparing saline groups in series A, B or C with the control or with one another. In Series A, when NAC was administered 60 minutes before the occlusion, NAC pretreatment significantly diminished infarction volume (n=12) in comparison to the saline injected animals. The average reduction of the total calculated infarction volume was 43% versus saline treated group (p=0.0007, n=10) (FIGS. 5A and 6). In series B, when NAC was injected 30 minutes before the occlusion, similar protection was detected in the NAC treated group (n=11). The average protection was 34% versus the saline (p=0.0072, n=10) (FIGS. 5B and 6). In series C, when NAC was administered after the reperfusion, mimicking the timing of the therapeutic interventions with thrombolysis, the total volume of infarction was smaller in the NAC treated group (n=5), but the difference was not significant (p=0.84) versus the saline treated group. Interestingly, the total infarction volume in the saline group of this series was obviously smaller in comparison to the saline groups of series A and B, but did not reach significance in the 7 group analysis (p>0.2).

In series D, one BSO+saline treated animal died overnight due to infarction induced edema and herniation, so only 6 animals underwent volumetric analysis. In BSO treated, glutathione depleted animals, NAC failed to provide significant protection in comparison to the saline controls (p=0.72) in a separate analysis using Student's t-test, however due to the low animal count, the statistical power of that analysis is low. Repeated measures analyses of variance using infarct area and the ratio of infarct area to the hemisphere area (across slices) were also performed. These analyses yielded similar results.

The above data demonstrated that intravenous administration of 400 mg/kg of NAC 30-60 minutes before the transient occlusion significantly diminished infarction volume compared to saline infused animals (p=0.05). The neuroprotection provided by NAC prophylaxis was detectable in comparison to the untreated control animals as well, but the difference was significant only for Series A (0.0055). However, it is noted that the experimental setting and fluid load were not identical in the untreated and infused animals. Regarding the calculated total infarction volume, there was no significant difference whether the NAC was administered 30 or 60 minutes before the occlusion.

When NAC was administered at the time of the MCA reperfusion, reproducing the timing of thrombolytic treatment of human strokes, no significant protection was observed in the NAC versus the saline treated animals. Surprisingly animals in both groups developed significantly smaller infarction than the other groups. Without wishing to be bound by a particular theory, it is hypothesized that the rapid hypervolemic hemodilution (with NAC or saline) may provide this beneficial effect. Both NAC and saline were administered as 2500 μl volume intravenous injection in 5 minutes, dropping the hematocrit from 50% to 30% according to our measurements. Hemodilution is reported to diminish brain reperfusion injury (Belayev et al., J. Neurosurg 87: 595-601,1997).

Experiments using 3 days of pretreatment with BSO in order to deplete glutathione levels (Neuwelt et al., Cancer Res. 61: 7868-74, 2001) showed no significant difference in NAC and saline treated animals (p=0.73), although the total infarction volume was smaller in NAC treated animals. These data suggest partial involvement of a glutathione sensitive pathway in the neuroprotective effect on NAC. However, BSO pretreatment did not result in higher infarction volume in comparison to other groups, but BSO administration causes intensive fluid overload (approximately 14 ml per day for 3 days) and weight loss of the animals, which makes direct comparison to other series difficult.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method for preventing or reducing an ischemia-reperfusion injury, comprising administering to a subject in need thereof an effective amount of a free radical scavenger intra-arterially or intravenously prior to, concurrently with, or following reperfusion.

2. The method according to claim 1 wherein the free radical scavenger is administered in an amount sufficient for the serum concentration of the scavenger to be from about 1 mM to 40 mM.

3. The method according to claim 1 wherein the free radical scavenger is a thiol-containing compound.

4. The method according to claim 1 wherein free radical scavenger is selected from the group of consisting of N-acetylcysteine, sodium thiosulfate, glutathione ethyl ester, glutathione, D-methionine, cysteamine, cystamine, aminopropylmethylisothiourea, and Ethyol.

5. The method according to claim 1 wherein the free radical scavenger is N-acetylcysteine.

6. The method according to claim 1 wherein the ischemia-reperfusion injury is an infarction, and wherein the volume of the infarction is reduced.

7. The method according to claim 6 wherein the infarction is in the brain.

8. The method according to claim 1 wherein the ischemia-reperfusion injury is a cerebral injury.

9. The method according to claim 1 wherein the ischemia-reperfusion injury is cerebral hemorrhage.

10. The method according to claim 1 wherein the ischemia-reperfusion injury is associated with a cardiopulmonary bypass procedure.

11. The method according to claim 10 wherein the cardiopulmonary bypass procedure is performed using a cardiopulmonary bypass circuit.

12. The method according to claim 10 wherein the cardiopulmonary bypass procedure is coronary artery bypass grafting.

13. The method according to claim 10 wherein the ischemia-reperfusion injury is cognitive dysfunction.

14. The method according to claim 1 wherein the free radical scavenger is administered intravenously.

15. The method according to claim 1 wherein the free radical scavenger is administered intra-arterially.

16. The method according to claim 15 wherein the free radical scavenger is administered via carotid artery.

17. The method according to claim 1 wherein the free radical scavenger is administered prior to reperfusion.

18. The method according to claim 8 wherein the free radical scavenger is delivered to the central nervous system.

19. The method according to claim 10 wherein the free radical scavenger is administered at least 15 minutes prior to the bypass procedure.

20. The method according to claim 10 wherein the free radical scavenger is administered at least 15 minutes prior to the bypass procedure.

21. The method according to claim 10 wherein the free radical scavenger is administered at least 30 minutes prior to the bypass procedure.

22. The method according to claim 10 wherein the free radical scavenger is administered at least 60 minutes prior to the bypass procedure.

23. The method according to claim 10 wherein the free radical scavenger is administered at least 90 minutes prior to the bypass procedure.

24. The method according to claim 1 wherein the free radical scavenger is administered in an amount sufficient for the serum concentration of the scavenger to be from about 3 mM.

25. The method according to claim 1 wherein the free radical scavenger is administered in an amount sufficient for the serum concentration of the scavenger to be from about 10 mM.
